(19) United States
(12) Patent Application Publication
(10) Pub. No.: US 2002/0146137 A1
(43) Pub. Date: Oct. 10, 2002

(54) Method for individualizing a hearing aid

(75) Inventors: Volker Kuhnel, Mannedorf (CH); Andreas Von Buol, Zurich (CH)

Correspondence Address: PEARNE & GORDON LLP, 526 SUPERIOR AVENUE EAST, SUITE 1200, CLEVELAND, OH, 44114-1484, US

(73) Assignee: Phonak AG

(21) Appl. No.: 09/829,700

(22) Filed: Apr. 10, 2001

(51) Int. Cl. H04R 29/00; A61B 5/00

(52) U.S. Cl. 381/60; 600/559

(57) ABSTRACT

This invention relates to a method for the individualized adaptation of a hearing aid to a person. The method consists basically of the measurement and quantification by parameters of the loudness perception of the individual, weighted by a first factor. Also weighted is a standardized normal loudness perception and its parameters by a second factor. Finally, the weighted loudness perceptions and their parameters are used for determining the optimal settings of the hearing aid for the individual concerned. The advantage of the method according to this invention lies in the fact that it permits significantly better adaptation of the hearing aid to the individual person.

This invention relates to a method for individualizing a hearing aid.

Successfully fitting a hearing-impaired individual with a hearing aid that is to correct for the hearing impairment is a critical factor which, among other things, determines the person's acceptance of the hearing aid. In this context it is not only the nature and degree of the hearing impairment that is of significance but there are various other factors as well, for instance the person's particular perception of loudness levels.

The disclosure document of the European patent application number EP-A2-0 661 905 describes one such method for fitting a person with a hearing aid. That earlier method addresses the correction of the damaged psycho-acoustic perception of an individual by a parameter adjustment in the hearing aid. The targeted correction uses as a reference value the statistically determined average auditory perception of persons with normal hearing.

The above-mentioned patent disclosure further indicates that a loudness scaling procedure is employed for establishing a dynamic-compression default setting in the hearing aid. This permits on an individualized basis the determination of the acquisition level in the case of inner-ear damage, and thus equally individualized compensation. Additional reference is made in this connection to the publications by Kiessling, Kollmeier and Diller titled “Outfitting and Rehabilitation with Hearing Aids” (1997, Thieme, Stuttgart, New York) and by Thomas Brand titled “Analysis and Optimization of Psychophysical Procedures in Audiology” (Oldenburg: Library and Information System of the University, 2000-148 pp., Oldenburg, Diss., Univ., 1999, ISBN 3-8142-0721-1).

The loudness standard serving as a reference was established based on a group of persons with normal hearing, employing, where possible, the same procedure for determining that standard auditory function that is used in the specific individual measurements.

Various investigations have made it evident that auditory perception can differ significantly even within the loudness standard. A summary of the data established is contained in the publication by C. Elberling titled “Loudness Scaling Revisited” (J Am Acad Audiol 10, pp 248 to 260, 1999).

It is therefore the objective of this invention to introduce a method for providing settings in the hearing aid which permit an improved adaptation of hearing aids to the loudness perception of the individual.

This is accomplished by means of the procedure specified in claim 1, with subsequent claims specifying desirable implementation versions of the invention.

The advantages offered by this invention are as follows: Both the auditory perception of the individual and the statistical average auditory perception of hearing-impaired persons as a function of their loss of hearing as well as the standard auditory perception of persons with normal hearing are taken into account in defining the settings of a hearing aid, appropriately weighted on the basis of data reliability, the result being optimized target parameters for adjusting the settings of the individual's hearing aid, and thus improved hearing of the individual. In other words, this invention has made it possible to obtain a target loudness level which is optimized for the loudness perception of the individual.

The following description explains this invention in more detail with the aid of drawings in which

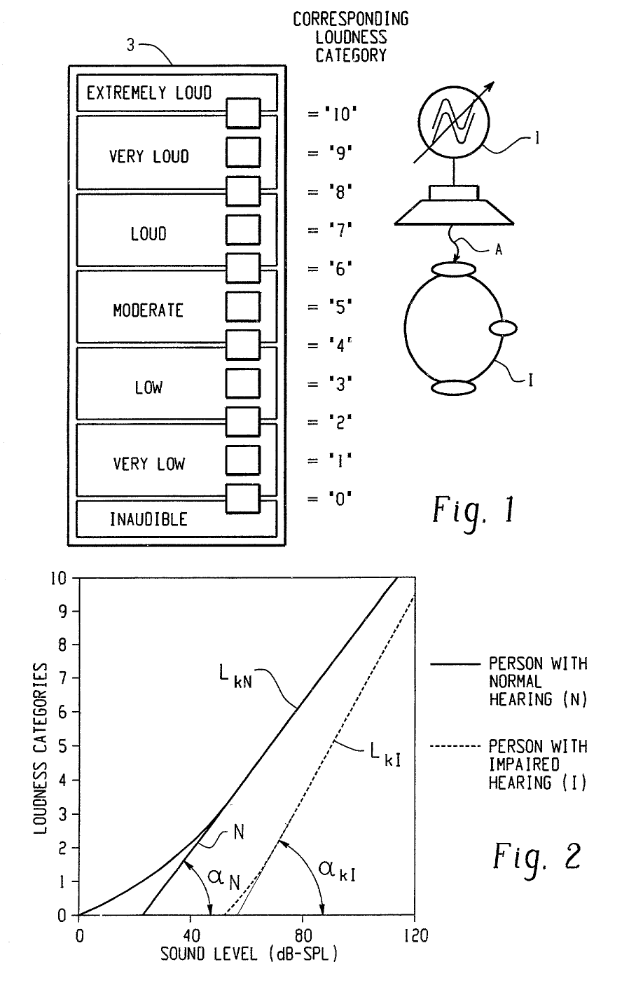

FIG. 1 is a schematic illustration of a quantification unit serving to quantify an individually perceived loudness level;

FIG. 2 indicates the loudness level perceived by a person with normal hearing and, respectively, by a person with impaired hearing, as a function of volume and at a specific frequency;

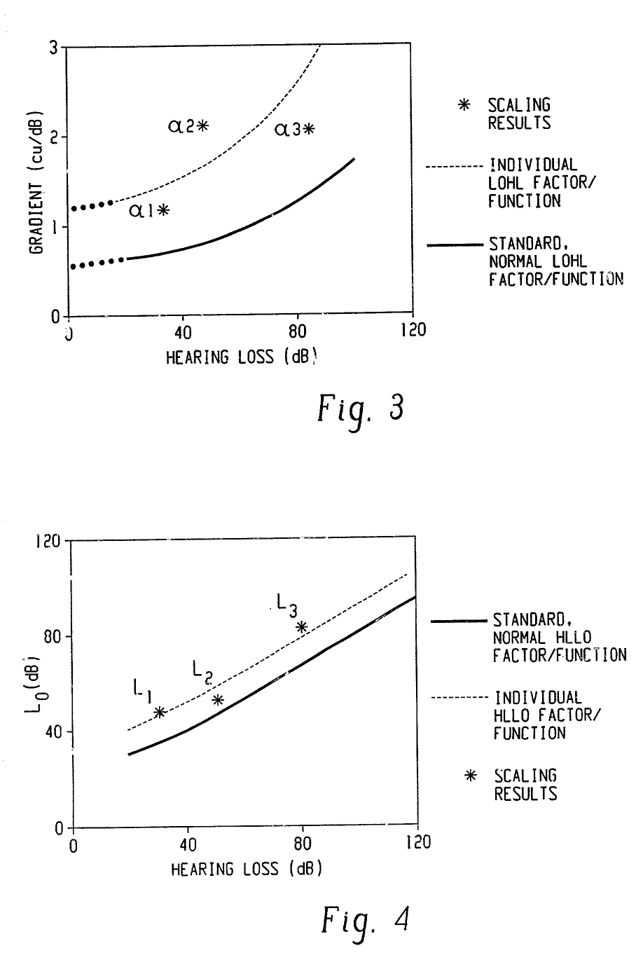

FIG. 3 shows the loudness correction as a function of the loss of hearing (HVLS/LOHL function) of a hearing-impaired person; and

FIG. 4 shows the level for loudness=0 as a function of hearing loss (HVLO/HLLO function) for a hearing-impaired person.

As is already evident from the introductory statements, the invention provides the possibility of an individualized and consequently better adjustment of hearing aids by virtue of the fact that the hearing-aid setting takes into account deviations attributable to inaccurate measurements as well as scattered values resulting from different individual loudness perceptions, with appropriately weighted individually established parameters as well as the standard loudness perception contributing to the definition of optimal adaptation. The term “optimal adaptation” in this case refers in particular to the setting of a balanced compression pattern and of the amplification, i.e. the frequency-dependent input/output characteristics of the hearing aid.

In terms of the compression, this is accomplished in particular by plotting the specific gradients of the individual scaling results as a function of the loss of hearing and approximating them by a specific HVLS/LOHL function, i.e. by the gradient of the loudness factor as a function of the hearing loss HV/HL. The individual HVLS/LOHL function when compared to the average hearing-impaired HVLS/LOHL function permits the determination of a factor which describes the loudness sensitivity of the individual in comparison with the standard.

In terms of the amplification, this is accomplished by plotting the specific levels L0 of the individual scaling results as a function of the hearing loss and approximating them by a specific HVL0/HLL0 factor, where the level for loudness=0 as a function of the loss of hearing HV/HL. The individual HVL0/HLL0 factor, compared to the average HVL0/HLL0 factor of the hearing-impaired, permits the determination of an offset which describes the mean value of the difference in the abscissa of the loudness function of the individual in comparison with the standard.

The following is a step-by-step explanation of the procedure for the adaptation of a hearing aid.

First, an audiogram is prepared. For a potential wearer of a hearing aid this is done by measuring the hearing thresholds for pure sounds at different frequencies. The increments of these audible limits are expressed and plotted as hearing loss in dB for each frequency and at certain frequency intervals. The audiogram thus allows for the determination of the auditory range in which there is a hearing loss. The audiogram also establishes data sampling points, meaning individual frequencies, at which loudness scaling is subsequently performed in the manner described next.

The loudness “L” is a psycho-acoustic variable which indicates how “loud” an acoustic signal is perceived by an individual.

In the case of natural acoustic signals which are always broad-band signals, the loudness does not necessarily match the physically transmitted energy of the signal. A psycho-acoustic analysis of the impinging acoustic signal takes place in the ear within individual frequency bands, the so-called critical bands. The loudness is determined by a band-specific processing of the signal and an inter-band superposition of the band-specific processing results, known as “loudness summation”. These basic principles were described in detail by E. Zwicker in “Psychoacoustics”, Springer-Verlag Berlin, academy edition, 1982.

It has been found, however, that loudness must be viewed as one of the most essential psycho-acoustic variables determining acoustic perception.

One possibility to use the loudness individually perceived in response to selected acoustic signals as a variable for further processing is offered by the method schematically illustrated in FIG. 1 and described for instance by O. Heller in “Auditory Range Audiometry Employing the Categorization Method”, Psychological Articles 26, 1985, or by V. Hohmann in “Dynamics Compression for Hearing Aids, Psychoacoustical Fundamentals and Algorithms”, thesis at the Univ. of Gottingen, VDI-Verlag, Series 17, No. 93, or by Thomas Brand in “Analysis and Optimization of Psychophysical Procedures in Audiology”, (Oldenburg: Library and Information System of the University, 2000-148 pp., Oldenburg, Diss., Univ., 1999, ISBN 3-8142-0721-1). According to that method, a person I is exposed to an acoustic signal A which can be varied in a generator 1 in terms of its spectral composition and its transmitted sound pressure level. The person I analyzes i.e. “categorizes” the acoustic signal A just heard by means of an input unit 3 within for instance eleven loudness steps or categories as illustrated in FIG. 1. These steps are assigned numerical weights for instance from 0 to 10.

By means of this approach it is possible to measure or quantify the specific loudness perceived. According to this invention, the process, hereinafter referred to as loudness scaling, is performed at a minimum of one and preferably at three different frequencies or data sampling points.

In FIG. 2 the loudness L, registered by category scaling per FIG. 1, is expressed as a function of the mean sound pressure level in dB-SPL for a sinusoidal signal of frequency fₖ. As is evident from the pattern in FIG. 2, the loudness K_{kN} of the standard in the graph chosen increases in nonlinear fashion with the signal level; in a first approximation the slope for persons with normal hearing is expressed for all critical bands by the regression line indicated as N in FIG. 2 with a gradient α_{N} in [categories per dB-SPL].

It is quite evident from this illustration that the model parameter α_{N} corresponds to a nonlinear amplification which for persons with normal hearing is approximately the same in each critical frequency band, whereas for hearing-impaired persons the determination must be made using α_{kT} for each frequency or frequency band.

The straight line with the gradient α_{kT} serves to approximate the nonlinear loudness function at frequency fₖ by means of a regression line.

In FIG. 2, L_{kT} indicates the typical pattern of loudness L_{T} of a hearing-impaired person at a frequency of fₖ.

A comparison of the curves L_{kN} and L_{kT} shows that the curve of a hearing-impaired person displays a greater offset (Lₒ) relative to zero and has a steeper slope than the standard curve. The greater offset corresponds to a higher audible limit or hearing threshold; the phenomenon of the invariably steeper loudness curve is referred to as loudness “recruitment” or acquisition and reflects a higher a-parameter.

As pointed out further above, loudness scaling is performed at a minimum of one and preferably at three reference or data sampling points, i.e. at one or several different frequencies. Based on these reference values a so-called HVLS/LOHL factor is established by plotting the gradients of the loudness factor α₁, α₂, α₃, . . . as a function of hearing loss HV/HL in dB.

FIG. 3 shows an HVLS/LOHL function for a hearing-impaired person, with the individual HVLS/LOHL function, represented by the dashed line, established via three data sampling points for building a suitable model as explained below.

The following model has been found to be particularly useful in determining the gradient α as a function of hearing loss HV/HL (for hearing loss between 20 dB and 100 dB):

log₁₀(α)=aₐ×HV/HL+bₐ×log(HV/HL)+VP_{consta} for 20 dB<HV/HL<100 dB,

where

α=gradient of the loudness function,

HV/HL=hearing loss in dB,

aₐ, bₐ=constant function parameter, and

VP_{consta}=the individual function parameter which adapts the HVLS/LOHL factor to the data sampling points α₁, α₂, α₃, . . .

It should be mentioned at this juncture that, having been extrapolated from several data sampling points, the individual HVLS/LOHL factor illustrated in FIG. 3 shows less dispersion-related deviation than do the sampling points by themselves, thus providing a better reflection of changes in individual perception. Although it would be possible to obtain the targeted reference settings for the hearing aid already on the basis of this individual HVSL/LOHL factor, to determine the gradient α at 0 dB hearing loss by extrapolation (dotted curve in FIG. 3) and to set the hearing aid accordingly, it has been found that the setting of the hearing aid can be substantially improved if data on the healthy ear are also included in the equation. According to the invention the normal loudness perception should be used as a reference for determining the individually needed compression at 0 dB hearing loss. In the process, according to the invention, the fact is taken into account that even the loudness perception of persons with normal hearing tends to vary to a more than negligible extent.

As a preferred solution for including the normal-loudness factor, a mean value is established between the individual gradient α at 0 dB hearing loss, determined by measurements and by extrapolation, and the normal-loudness gradient, weighting the values based on their expected dispersion both for the individual gradient α at 0 dB hearing loss and for the normal-loudness gradient. Weighting the individual scaling data as a function of their respective quality and of the number of measuring points for the various scaling functions and the number of scaling operations themselves has proved to be useful. For individual scaling data of average quality at three frequencies, a weighting of the individual gradient α at 0 dB hearing loss by a factor of ⅔ and a weighting of the normal-hearing gradient α_{N} by a factor of ⅓ can lead to an exceedingly good adaptation of the hearing aid.

Similar to the gradient α for the loudness function, the abscissa section L₀ of the loudness factor in conjunction with the hearing loss information established in the audiogram permits the determination of an optimum band-specific amplification.

As pointed out further above, loudness scaling is performed at a minimum of one and preferably at three reference or data sampling points, i.e. at one or several different frequencies. Based on these data points the HVL0/HLL0 factor is established by plotting the abscissa sections for the loudness factor L₀₁, L₀₂, L₀₃, . . . as a function of hearing loss HV/HL in dB.

FIG. 4 shows the HVL0/HLL0 factor for a hearing-impaired person with the individual HVL0/HLL0 function, represented by the dashed line, established via three data sampling points for building a suitable model as explained below.

The following model has been found to be particularly useful in determining L₀ as a function of hearing loss HV/HL (for hearing loss between 20 dB and 100 dB):

L₀=α_{L}×HV/HL+b_{L}×log(HV/HL)+VP_{constL} for 20 dB<HV/HL<100 dB,

where

L₀=level of loudness=0,

HV/HL=hearing loss in dB,

a_{L}, b_{L}=constant function parameter, and

VP_{constL}=individual function parameter which adapts the HVL0/HLL0 function to the data sampling points L₀₁, L₀₂, L₀₃, . . .

It should be mentioned at this juncture that, having been extrapolated from several data sampling points, the HVL0/HLL0 factor illustrated in FIG. 4 shows less dispersion-related deviation than do the sampling points by themselves, thus providing a better reflection of changes in individual perception. Although it would be possible to obtain the targeted reference settings for the hearing aid already on the basis of this individual HVL0/HLL0 factor, to determine the level L₀ at 0 dB hearing loss by extrapolation (dotted curve in FIG. 3) and to set the hearing aid accordingly, it has been found that the setting of the hearing aid can be substantially improved if, similar to the gradient a, data on the healthy ear are also included in the equation. According to the invention the standard i.e. normal loudness perception should be used as a reference for determining the individually needed compression at 0 dB hearing loss. In the process, according to the invention, the fact is taken into account that even the loudness perception of persons with normal hearing tends to vary to a more than negligible extent.

As a preferred solution for including the normal-loudness factor, a weighted mean value is established between the individual level L₀ at 0 dB hearing loss, determined by measurements and by extrapolation, and the normal level L₀, weighting the values based on their expected dispersion both for the individual level L₀ at 0 dB hearing loss and for the normal level L₀. For the level L₀ as well, similar to the gradient of the loudness factor, weighting the individual scaling data as a function of their respective quality and of the number of measuring points for the various scaling functions and the number of scaling operations themselves has proved to be useful.

For individual scaling data of average quality at three frequencies, a weighting of the individual level L₀ at 0 dB hearing loss by a factor of ⅓ and a weighting of the normal-level L₀ by a factor of ⅔ can lead to an exceedingly good adaptation of the hearing aid.

1. Method for individualizing a hearing aid in adaptation to the loudness perception of the individual, said method consisting of the following:
Measurement and quantification by parameters of the loudness perception of the individual, weighted by a first factor;
Weighting of a normal loudness perception and its parameters by a second factor and use of the weighted loudness perception and its parameters for adjusting the hearing aid.

2. Method as in claim 1, whereby the compression and/or amplification is/are adjusted in the hearing aid, for which purpose the compression and, respectively, the amplification are each determined as a function of the frequency.

3. Method as in claim 2, whereby, for determining the compression, the loudness perception of the individual is quantified by means of a HVLS/LOHL factor which is determined by loudness scaling at a minimum of one frequency.

4. Method as in claim 3, characterized in that the HVLS/LOHL factor is modeled using the equation
log₁₀(α)=aₐ×HV/HL+bₐ×log(HVHL)+VP_{consta}
where
α=gradient of the loudness function,
HV/HL=hearing loss in dB,
aₐ, bₐ=constant function parameter, and
VP_{consta}=the individual function parameter which adapts the HVLS/LOHL factor to the data sampling points α₁, α₂, α₃, . . .
and that VP_{consta} is determined on the basis of a loudness scaling performed at a minimum of one frequency and preferably at three different frequencies.

5. Method as in claim 2, whereby, for determining the amplification, the loudness perception of the individual is quantified by means of an HVL0/HLL0 factor which is defined by loudness scaling at a minimum of one frequency.

6. Method as in claim 5, characterized in that the HVL0/HLL0 factor is modeled using the equation
L₀=a_{L}×HV/HL+b_{L}×log(HV/HL)+VP_{constL,}
where
L₀=level of loudness=0,
HV/HL=hearing loss in dB,
a_{L}, b_{L}=constant function parameter, and
VP_{constL}=individual function parameter which adapts the HVL0/HLL0 function to the data sampling points L₀₁, L₀₂, L₀₃, . . .
and that VP_{constL} is determined on the basis of a loudness scaling performed at a minimum of one frequency and preferably at three different frequencies.

7. Method as in one or several of the claims 3 to 6, whereby the hearing loss is used for determining the frequencies at which loudness scaling is performed.

8. Method as in one or several of the preceding claims, characterized in that the value of the weighted factors depends on the assumed and/or determined accuracy of the loudness scaling data.

9. Method as in claim 8, characterized by the selection of a value of ⅔ for the first factor and of a value of ⅓ for the second factor.
